(19) United States
(12) Patent Application Publication
(10) Pub. No.: US 2008/0228233 A1
(43) Pub. Date: Sep. 18, 2008

(54) INSTRUMENT FOR MANIPULATING SPINAL IMPLANT SYSTEM

(76) Inventors: Jeffrey Hoffman, Marquette, MI (US); Gregory Berrevoets, Skandia, MI (US); Maria Norman, Negaunee, MI (US)

Correspondence Address: FITCH EVEN TABIN AND FLANNERY, 120 SOUTH LA SALLE STREET, SUITE 1600, CHICAGO, IL, 60603-3406, US

(21) Appl. No.: 12/030,102

(22) Filed: Feb. 12, 2008

Related U.S. Application Data
(60) Provisional application No. 60/889,449, filed on Feb. 12, 2007.

(51) Int. Cl. A61F 5/00

(52) U.S. Cl. 606/86 A

(57) ABSTRACT

Instruments are provided herein for reducing the lateral distance between a spinal rod anchored to a spine and a coupling device. The instruments permit a rod to be shifted into a coupling device that is not in alignment with other coupling devices secured to the rod because of rotational deformities causing one or more vertebrae to be shifted with respect to at least one other vertebra.

### CROSS-REFERENCE TO RELATED APPLICATIONS

This application claims priority to U.S. Provisional Application Ser. No. 60/889,449, filed Feb. 12, 2007, and entitled “Instrument for Manipulating Spinal Implant System,” which is hereby fully incorporated by reference as if set forth herein.

### FIELD OF THE INVENTION

The invention relates to an instrument for manipulating and securing a spinal rod relative to a spine and, more particularly, to an instrument for securing a spinal rod to one or more coupling devices fixed to the spine.

### BACKGROUND OF THE INVENTION

In a number of surgical procedures, implant devices are utilized to promote the healing and repair of various parts of the human body. In some cases, implant devices secure bones or bone segments relative to each other so that the bones themselves may heal, fuse, or be repositioned. For instance, two or more vertebrae of the spinal column may be linked together by a plate or an elongated rod member in order to prevent relative movement between the vertebrae. In addition, an elongated rod may be used to correct spinal deformities, including rotating or de-rotating one or more vertebrae relative to at least one other vertebra. For instance, in treatments for scoliosis, undesirable torsion of the spine is corrected by “de-rotating” one or more rotated, out-of-phase vertebrae to place them in proper rotational alignment with the other vertebrae.

Typically, implanting devices that secure bones or bone segments relative to each other involves securing a plurality of bone screws, hooks, or other fixtures to a plurality of respective bones. Then, each of the fixtures is secured relative to the others with an additional apparatus, such as a connecting rod. A pedicle screw and rod system is one such example that is commonly used to connect adjacent vertebrae together.

In order to align a series of vertebrae, a number of bone screws may be secured to or fastened along the vertebrae. Each screw may be integrally or rotatably attached to a coupling member, which includes physical structures for coupling a bone screw to a connecting rod. Often, the coupling member includes opposed, upstanding walls to form a yoke within which the connecting rod is retained. Each coupling member may be secured with, and relative to, at least one other coupling member with the spinal rod. A locking member, such as a locking cap, is locked into the coupling member to lock the spinal rod relative to the coupling member.

A number of methods may be used to lock a spinal rod within a coupling member. Traditional locking caps require at least partial rotation of a cap relative to a coupling member in order to loosely secure the cap to the coupling member. Further rotation of the cap provides additional locking force that compresses the rod into the coupling member and locks it into place. Many pedicle screws, for instance, utilize a threaded locking cap that engages threads on the interior or exterior of the yoke so that rotation of the cap relative to the yoke results in linear movement of the locking cap toward the spinal rod. Threading the cap incrementally into the coupling member causes an incremental increase in the force securing the spinal rod. When the cap is rotated enough times, a clamping force is provided to secure the rod between the yoke and the locking cap. Other locking devices (such as in U.S. Pat. Nos. 5,084,049 to Asher; 6,565,565 to Yuan; and 6,755,829 to Bono) include discrete flanges or slots that may be lowered onto or into a coupling member and then twisted into place with a partial rotation to at least loosely capture a spinal rod within the coupling member. In such devices, the locking member will simply fall out of the coupling member unless the flanges or slots are rotated into contact with corresponding structures on the coupling member. Alternatively, a novel, linearly-inserted locking cap assembly has been provided in U.S. patent application Ser. No. 11/839,843, filed Aug. 16, 2006 (which is incorporated by reference as if fully described herein), wherein flexible portions of the walls of the coupling member or yoke flex outward and inward to capture a linearly-inserted cap in a snap-lock fit. In one device disclosed in that application, a cap is inserted into the yoke without rotation to a first snap-lock position within the yoke, at least loosely capturing the rod within the yoke. Further insertion may lead to one or more additional snap-lock positions. The locking cap may further be configured so that rotation of at least a portion of the locking cap assembly when received in the yoke to provide additional locking force, pushing a bottom surface of the cap assembly against the spinal rod and locking the rod into place.

When securing a rod to the spine, the fixation members implanted into the vertebrae may not be perfectly aligned for receipt of a straight rod. In such cases, the rod may be bent to conform to the fixation members, or the vertebrae may be rotated slightly to align the fixation members. However, in some applications, a spinal deformity is corrected by shifting and/or rotating the vertebrae to conform to a rod with a preselected shape. For instance, coupling members may be anchored along the vertebrae so that each coupling member is in relatively the same position with respect to its vertebra as other coupling members are to their respective vertebra, so that when the coupling members are aligned along a straight rod, the vertebrae are rotated and forced into alignment. In such cases, aligning the coupling members anchored to the vertebrae along the rod is often difficult, requiring a significant amount of torque to be applied to the patient's spine in order to rotate the coupling devices and their respective vertebrae into axial alignment. Maintaining the positioning of the coupling devices, locating a connecting rod into the aligned coupling devices, and locking the rod within the coupling members while the vertebrae are under significant torsional stress all presents further challenges.

There remains a need for an improved implant manipulation instrument that may apply the force required to draw a spinal rod laterally into alignment with a coupling member anchored to a vertebra. There also remains a need for a device that is able to secure and lock a rod into place within a coupling member anchored to a vertebra while counteracting torque created by undesirable rotation or other deformities of the spine.

### SUMMARY OF THE INVENTION

The present invention is related to devices and methods to aid in maneuvering and securing an implantable structure, such as a spinal rod, to a fixation device, such as a pedicle screw or vertebral hook.

An instrument is provided for reducing or eliminating the lateral distance between a coupling member (or yoke) fixed to a first vertebra and an adjacent spinal rod fixed to at least two other vertebra. The instrument comprises a clamp device for clamping the yoke fixed to the vertebra; a persuader arm pivotably connected to the clamp device that engages a spinal rod and manipulates the rod to locate it in the yoke secured by the clamp; and a drive device to axially insert a locking cap into engagement with the yoke to secure the spinal rod therebetween. The persuader arm is configured so that the rod may be positioned into the yoke without disengaging the persuader arm from the rod. The drive device may be configured to be inserted axially through the persuader arm in order to insert the locking cap into the yoke while the rod is contacting both the yoke and the persuader arm. The drive device is preferably an assembly including a component that axially advances the locking cap toward the yoke into one or more axial positions, and a component that rotates the cap into one or more rotational positions.

The instrument may include a ratchet mechanism capable of restricting the pivoting motion of the persuader arm away from the clamp device, but permitting pivoting of the persuader arm toward the clamp device, thereby allowing the rod to be pulled toward the yoke secured to the clamp device but preventing the rod from moving away from the yoke due to forces created by the shape and contortion of the spine.

An axial adjustment lock assembly may also be included in the instrument. The axial adjustment lock engages and disengages the persuader arm to control axial movement of the arm. When the axial adjustment lock assembly is disengaged, the persuader arm may be freely adjusted axially in order easily extend the persuader arm into engagement with the rod. Once the persuader arm has captured the rod, the axial adjustment lock assembly may be engaged to prevent free axial movement so that the persuader arm may manipulate the rod without being moved axially by torque from the spine. Preferably, the axial adjustment lock assembly provides for ratcheted retraction of the persuader arm so that the persuader arm may be incrementally retracted to move the spinal rod toward the coupling member secured to the instrument.

The disclosed instrument may be used, for instance, with a system for securing a spinal rod where a locking cap is linearly advanced into one or more locking positions within a yoke, and then rotated to provide one or more additional locking positions that further restrict movement of a rod received in the yoke.

### BRIEF DESCRIPTION OF THE DRAWINGS

FIG. 1 is a side elevation view of a portion of a spine in which one vertebra is rotated relative to the adjacent vertebrae;

FIG. 2 is a view of the instrument without the persuader arm inserted into the guide portion;

FIG. 3 is a view of the instrument with the persuader arm inserted into the guide portion;

FIG. 4 is a view of the instrument engaging a yoke laterally displaced from a spinal rod;

FIG. 5 shows the instrument clamping to a yoke laterally displaced from a spinal rod;

FIG. 6 shows the persuader arm being inserted into the instrument when clamped to a yoke;

FIG. 7 shows the persuader arm of the instrument being extended to capture a spinal rod adjacent to the yoke to which it is clamped;

FIG. 8 is a view of the persuader arm of the instrument capturing a spinal rod;

FIG. 9 shows the instrument having shifted a spinal rod to a position above the yoke to which the instrument is attached;

FIG. 10 shows the persuader arm traveling linearly to position a spinal rod into a yoke;

FIG. 11 is a cross-sectional view of the persuader arm positioning a spinal rod above a yoke;

FIG. 12 is a side elevation view of a drive device and locking cap being inserted into the persuader arm of the instrument;

FIG. 13 is a side elevation view of the drive device with a locking cap releasably secured thereto at the distal end;

FIG. 14 is a cross-sectional view of the instrument positioning the spinal rod in the yoke, with the drive device inserted coaxially into the persuader arm;

FIG. 15 shows the instrument with its clamp device in an open position, engaging the yoke;

FIG. 16 shows the instrument clamping the yoke;

FIG. 17 is a side cross-sectional view of the instrument before clamping;

FIG. 18 is a side cross-sectional view of the instrument clamping the yoke;

FIG. 19 is a rear cross-sectional view of the clamping device before the actuator activates the clamping mechanism;

FIG. 20 is a rear cross-sectional view of the clamping after the clamping mechanism is engaged;

FIG. 21 is an exploded view of the instrument, without the drive device for inserting the locking cap;

FIG. 22 is an exploded view of the central operating assembly;

FIG. 23 is a perspective view of the ratchet mechanism;

FIG. 24 is a rear cross-sectional view of the instrument showing part of the clamp device actuating mechanism;

FIG. 25 is a cross-sectional view of the central operating assembly;

FIG. 26 shows the axial lock mechanism for the persuader arm in a disengaged position;

FIG. 27 shows the axial lock mechanism for the persuader arm in an disengaged position;

FIG. 28 is a side elevation view of the engagement portion of the persuader arm;

FIG. 29 is a cross-sectional view of the engagement portion of the persuader arm;

FIG. 30 is a detailed view of the distal end of the drive device and a locking cap to be secured thereto;

FIG. 31 is an exploded view of the drive device, showing the distal ends of the inner and outer shaft members; and

FIG. 32 is an exploded view of the drive device, showing the proximal ends of the inner and outer shaft members.

### DETAILED DESCRIPTION OF THE PREFERRED EMBODIMENTS

In one form, the instrument disclosed herein has a central housing containing a ratchet mechanism or assembly, with a ratchet handle and clamp device extending from the housing. A persuader arm is provided and configured for pivotal movement by operation of the ratchet handle. The persuader arm may be releasably secured to, and preferably inserted coaxially into, the ratchet handle. The clamp device of the instrument is configured to clamp to a coupling member, for instance the yoke of a bone screw, when the coupling member is anchored to a bone. The persuader arm is configured to extend from the instrument to capture a spinal rod, and to maneuver the spinal rod toward the clamp device in order to position the rod in the coupling member or yoke clamped within the clamp device.

Once a surgeon has secured the instrument to the yoke, the clamp device or member holds the yoke with sufficient clamping force so that the instrument is secured to the spine and need not be held in place by the surgeon.

During operation, the persuader arm captures a spinal rod that is mounted to the spine in a position laterally spaced from the yoke. The ratchet handle and persuader arm are pivoted with respect to the clamp device in order to maneuver the rod toward the clamp device. The ratchet assembly allows this pivoting to be accomplished incrementally, advantageously permitting additional surgical steps to be taken at intervals during manipulation of the rod. The persuader arm may also be retracted incrementally toward the instrument, preferably by turning a threaded portion of the persuader arm within a guide portion.

The persuader arm is retracted and pivoted, drawing the rod toward the instrument, until the rod is positioned axially above an opening in the yoke for receiving the spinal rod. The persuader arm is then incrementally advanced toward the yoke until the rod is positioned in the yoke. Preferably without disengaging the persuader arm from the rod, a drive device may be used to axially insert a locking cap into engagement with the yoke in order to lock the rod in place.

Advantageously, the steps of laterally moving the rod and the yoke, positioning the rod within the yoke, and locking the rod to the yoke may all be accomplished using a single instrument and without disengaging the rod between the positioning and locking steps.

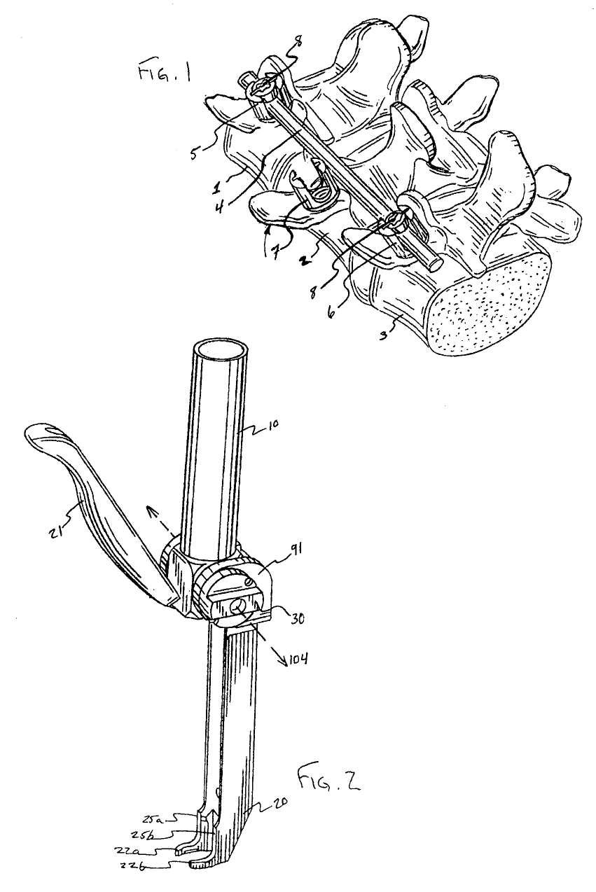

The instrument 100 described herein is especially useful for de-rotating a spinal column experiencing rotational deformity, as in FIG. 1. A rotated intermediate vertebra 2 is rotated or shifted with respect to two adjacent vertebrae 1 and 3. Vertebrae 1 and 3 are connected by a rigid spinal rod 4 captured by fixation members in the form of bone screw assemblies having yokes 5 and 6 mounted to and extending dorsally from vertebrae 1 and 3, respectively. The spinal rod 4 connecting yokes 5 and 6 is depicted as straight, having an axis parallel to the axis of the partial spinal column, and connecting two yokes 5 and 6 that are axially aligned along the spine to permit connection by the spinal rod 4. However, the yokes 5 and 6 need not be perfectly axially aligned, and the spinal rod 4 may experience some bending in order to be disposed in yokes 5 and 6. Yokes 5 and 6 may be any type of top loading bone screws designed to receive a spinal rod. The spinal rod 4 is held in place in the yokes 5 and 6 by locking caps 8.

An intermediate yoke 7, located axially between screw assemblies 5 and 6 along the axis of the spinal column and mounted to the intermediate vertebra 2, is out of phase with respect to the two adjacent yokes. Torsion of the intermediate vertebra 2 as a result of one or more spinal deformities has resulted in a lack of axial alignment between the intermediate vertebra 2 and adjacent vertebrae 1 and 3. The intermediate yoke 7 therefore is out of alignment with yokes 5 and 6, despite being anchored in the same relative position along its associated vertebra as adjacent yokes 5 and 6 to their respective vertebrae. The positioning of the intermediate yoke 7 is such that aligning the yoke 7 axially with adjacent yokes 5 and 6 will consequently shift the intermediate vertebra 2 roughly into axial and rotational alignment with the adjacent vertebrae 1 and 3.

With continued reference to FIG. 1, connecting the intermediate yoke 7 to the spinal rod 4 requires significant shifting of the rod 4, since the entire lateral distance between the rod 4 and the intermediate yoke 7 must be eliminated by overcoming the torsion between vertebrae 1, 2, and 3. The rod 4 and intermediate yoke 7, both of which are mounted to the spine, may be captured by an instrument 100 disclosed herein and used as anchor points by the instrument 100, which provides a lever for manipulating and shifting the yoke 7 and rod 4 together to eliminate any lateral distance therebetween and positioning the rod within the yoke.

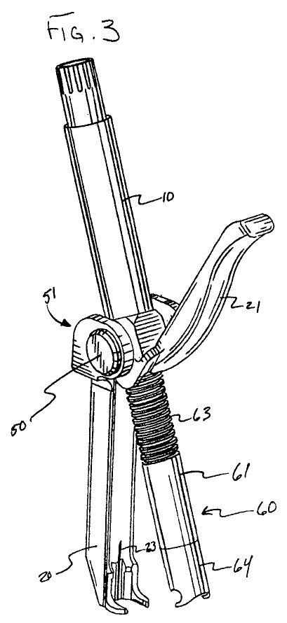

A perspective view of one embodiment of the instrument 100 is shown in FIG. 2. The instrument 100 contains a central housing 91 containing a central operating assembly. Extending from the central housing 91 are a ratchet handle 10 that pivots with respect to the housing, a clamp device 20, and a pivotable clamping actuator lever 21. The ratchet handle 10 and actuator lever 21 extend generally radially from the housing and pivot about an operating axis 104. In the illustrated embodiment, the clamp device 20 is integrally formed with the central housing 91. A ratchet dial 30 for operatively engaging a ratchet assembly 133, which when turned to a locked position inhibits pivoting between the ratchet handle 10 and the clamp device 20 in one direction, is located on the side of the housing 91 along the operating axis 104.

The clamp device 20 is a hollow elongated portion comprising at one end two parallel flexible legs 25a and 25b separated by a slit 23 (as shown in FIG. 3). Each flexible leg contains outwardly-extending arms 22a and 22b configured to receive and hold the yoke. The two parallel flexible legs 25a and 25b are shown formed integrally as part of a one-piece clamp device, but it will be understood that the clamp device may also comprise an assembly of separate structures that are movable with respect to each other.

Located on the central housing opposite the ratchet dial 30 is a button 50, as shown in FIG. 3, which may be pressed inward. The button controls an axial adjustment lock subassembly 59 that locks and unlocks an axial throughbore in the ratchet handle 10 (as shown in more detail in FIGS. 25-27). Pushing in the button 50 allows a persuader arm 60 to be slidably inserted through the ratchet handle 10, which serves as a guide device for the persuader arm 60. Alternatively, the guide device may be separate from the ratchet handle. The persuader arm 60 contains a threaded portion 63 along a shaft 61, and a distal engagement portion 64 configured to capture the spinal rod. Pivoting of the ratchet handle guide device 10 moves the persuader arm engagement portion 64 toward and away from the clamp device 20.

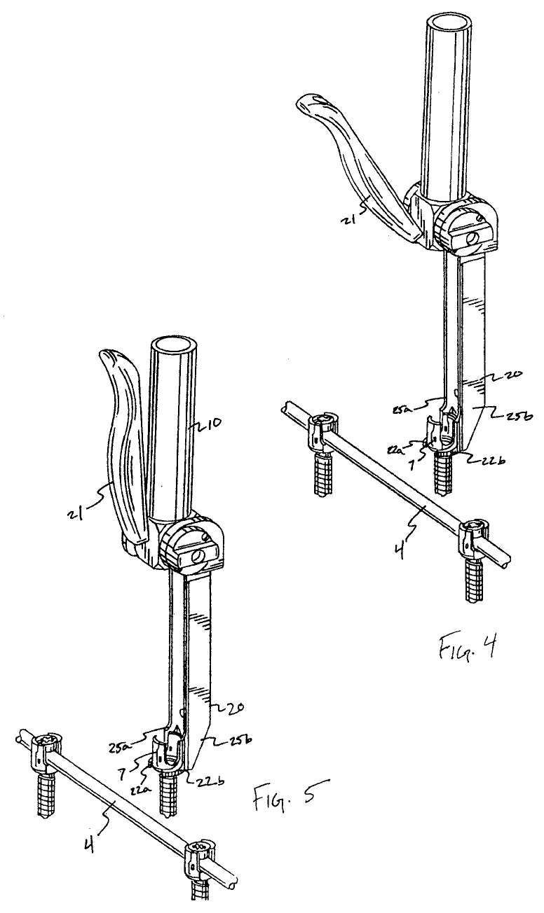

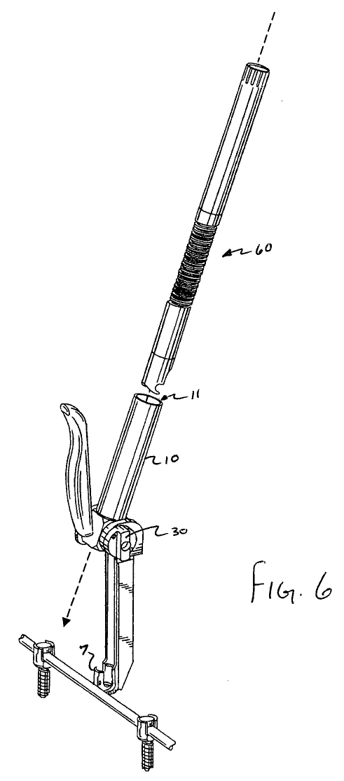

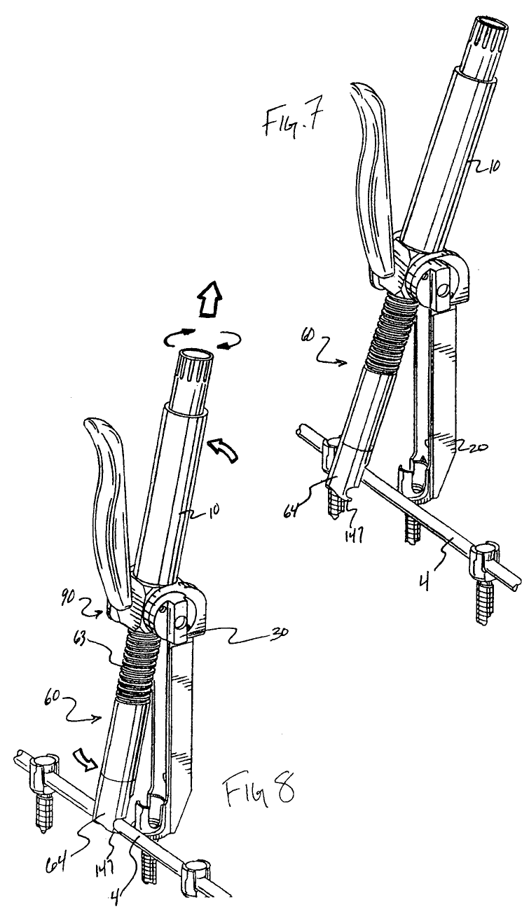

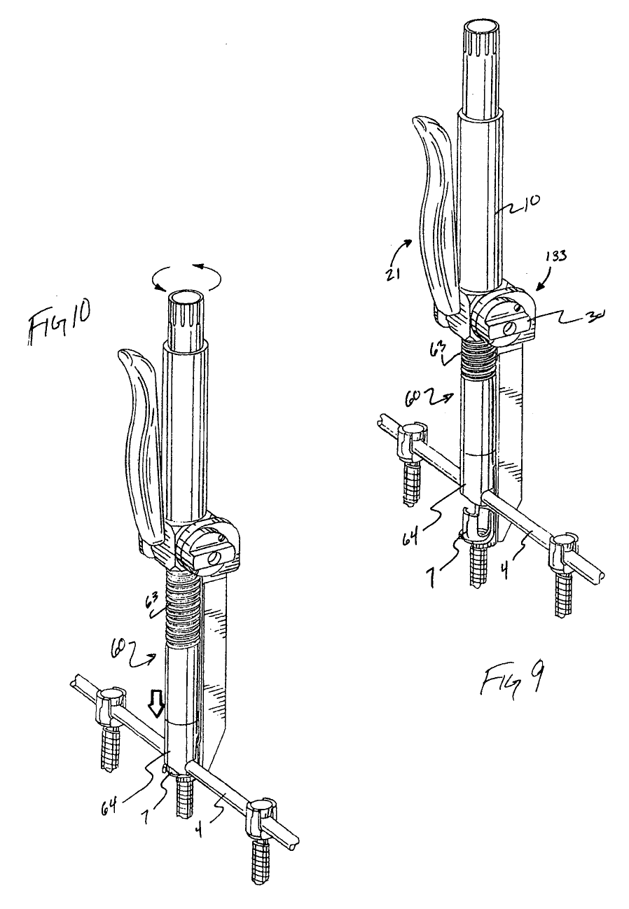

The instrument 100 is used to shift the laterally-positioned yoke 7 into alignment with the spinal rod 4, as shown in FIGS. 4-10. The instrument 100 is first secured to the implanted yoke 7 by engaging it with the arms 22a and 22b of the clamp device 20, as shown in FIG. 4. Once the clamp device is in place, the clamp actuator lever 21 is pivoted toward the ratchet handle 10 in order to clamp the yoke 7 in place. As will be described in detail later, movement of the actuator lever 21 causes the legs 25a and 25b of the clamp to flex inward, locking the yoke 7 in place between the clamp arms 22a and 22b.

Once the instrument 100 is clamped to the yoke 7, the persuader arm 60 is positioned within a guide portion, which in this case also forms the ratchet handle 10. The ratchet dial 30 is rotated to an unlocked position in order to allow the ratchet handle 10 to pivot while the persuader arm 60 is inserted therein. The persuader arm 60 is inserted axially through a throughbore 11 in the ratchet handle 10. The axial adjustment lock subassembly 59 is disengaged so that the persuader arm 60 may freely slide into the throughbore 11.

The persuader arm 60 is slidably extended toward the laterally spaced rod 4 as in FIG. 7. The head or engagement portion 64 of the persuader arm contains a hooked portion 147 in order to engage the spinal rod 4 at a side diametrically opposite the ratchet handle 10. The ratchet handle 10 is then pivoted in order to guide the persuader arm 60 so that the engaged portion 64 captures the rod 4, as in FIG. 8. An axial adjustment lock subassembly 59 within the central operating assembly 90 at this point may be permitted to engage the threaded portion 63 on the exterior of the persuader arm 60 to limit extension thereof (shown in FIGS. 25-27).

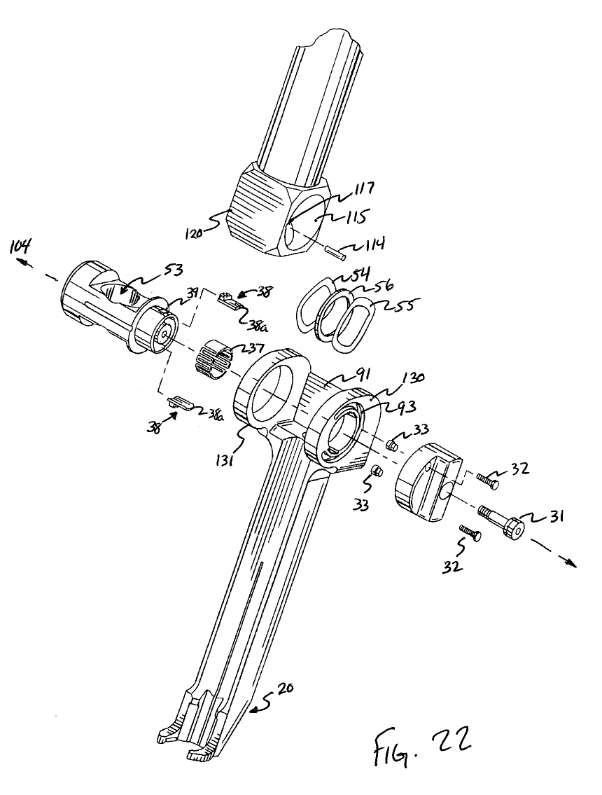

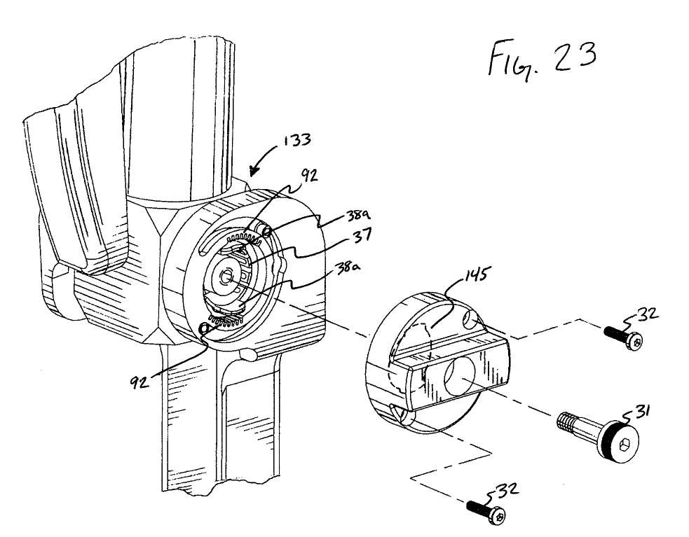

The ratchet dial 30 can be rotated to a locked position to prevent angular movement of the ratchet handle 10 away from a vertical position (which would allow the rod 4 to slide out of engagement with the engagement portion hooks 147). When in the locked position, the ratchet assembly 133 (shown in FIGS. 22-23) permits pivoting movement of the ratchet handle 10 in an upward arc only, so that the handle may be moved only toward a vertical position, thereby moving the persuader arm engagement portion 64 closer to the clamp device 20.

In most cases, axial movement of the persuader arm 60 is also desirable or necessary in order to raise the rod 4 above the level of the yoke 7 during pivoting. In order to position the rod 4 above the yoke 7, the persuader arm should be axially retracted through the ratchet handle 10 during pivoting. A drive mechanism is provided for incrementally moving the persuader arm 60 in an axial direction, comprising threads 63 on the exterior of the persuader arm 60 that are engageable with threads of the axial adjustment lock subassembly 59, described later in reference to FIGS. 25-27.

The persuader arm 60 is retracted and pivoted until the rod 4 is captured in the engagement portion 64 is located above the yoke 7, as in FIG. 9. Specifically, positioning of the rod is accomplished by rotating the persuader arm 60 as described above, and by ratcheting the persuader arm into a vertical position by squeezing the handle 10 and actuator lever 21 together, drawing the handle 10 upright into a vertical position. Since the handle 10 is locked in place by the ratchet assembly 133, the process of rotating the handle 10 into vertical alignment may be achieved incrementally, allowing additional surgical steps to take place at various stages of drawing the rod 4 toward the yoke 7. In fact, the surgeon may alternate between retracting and rotating the persuader arm 60 in order to carefully pull the spinal rod 4 toward the yoke 7 and de-rotate the spine. A cross section of the persuader arm 60 engaged to the spinal rod 4 is shown in FIG. 11.

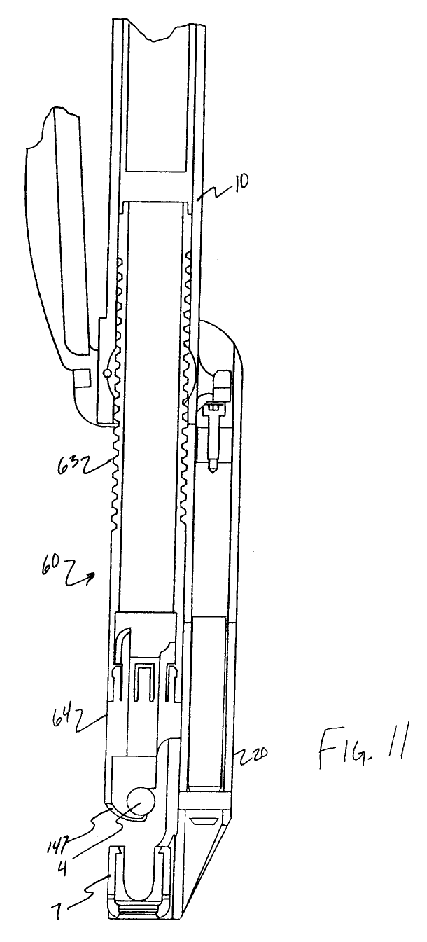

The spinal rod 4 is then shifted into the yoke 7, as shown by FIG. 10. Once the rod is drawn to a point above the yoke by axial retraction and pivoting of the persuader arm 60, the persuader arm 60 may be linearly advanced toward the yoke 7 by rotation in the opposite direction used to retract the persuader arm. The persuader arm engagement portion 64 is hollow and configured to be lowered over the yoke 7 to load the spinal rod 4 therein. The persuader arm engagement portion 64 engages the spinal rod 4 at axially spaced positions farther apart than the diameter of the yoke, so that no portion of the persuader arm engagement portion 64 is trapped between the rod 4 and yoke 7 as the rod is lowered into the yoke.

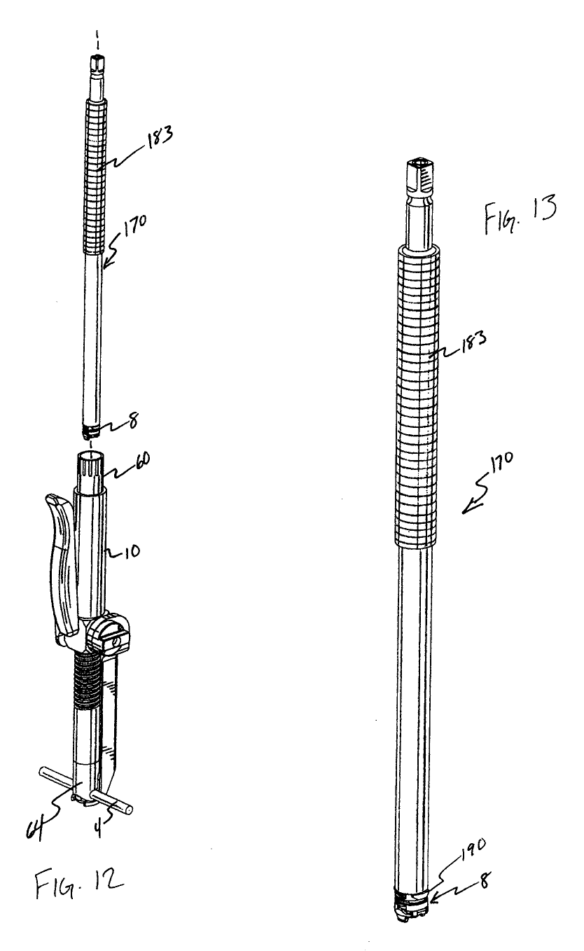

After the rod 4 has been positioned within the yoke 7, a drive device 170 may be inserted into an axial throughbore in the persuader arm 60 in order to drive the locking cap 8 into locking engagement with the yoke situated within the persuader arm engagement portion 64, as shown in FIG. 12. The drive device comprises an outer shaft member 171 in which an inner shaft member 172 is coaxially disposed, as seen in FIG. 14. The inner shaft member is rotatable within the outer shaft member 171, as seen in FIG. 14. The locking cap 8 is releasably secured to the distal end of the drive device 170. A bearing member 190 attached to the distal end of the drive device 170 holds the locking cap 8 into place.

The drive device 170 has a threaded portion 183, an outside diameter such that it can be inserted coaxially into the persuader arm 60, and into engagement with a threaded interior portion 184 of the persuader arm, as seen in FIG. 14. The drive device 170 is inserted axially into the persuader arm 60 until threads 183 on the drive device 170 engage threads 184 on the interior of the persuader arm. At that point, the outer shaft 171 of the drive device must be rotated in order to provide advancement of the drive device toward the yoke. A drive head portion 180 is provided at the proximal end and may be engaged with a wrench to advance the drive device downward toward the yoke 7.

Axially driving the drive device 170 and a locking cap 8 releasably secured thereto into engagement with the yoke 7 inserts the locking cap into the yoke 7, as seen in FIG. 14. In one form, linearly advancing the cap into the yoke at least partially locks the cap into the yoke. The locking cap and yoke may optionally be configured to enter into a plurality of locking positions as the locking cap 8 is axially inserted into the yoke, and the cap may also be configured to enter one or more locking positions as it is rotated relative to the yoke 7 after axial insertion. Rotation of the inner shaft member 172 causes rotation of the locking cap 8, and the locking cap 8 and yoke 7 may therefore also be configured so that rotation of the locking cap 8 within the yoke 7 achieves one or more locking positions.

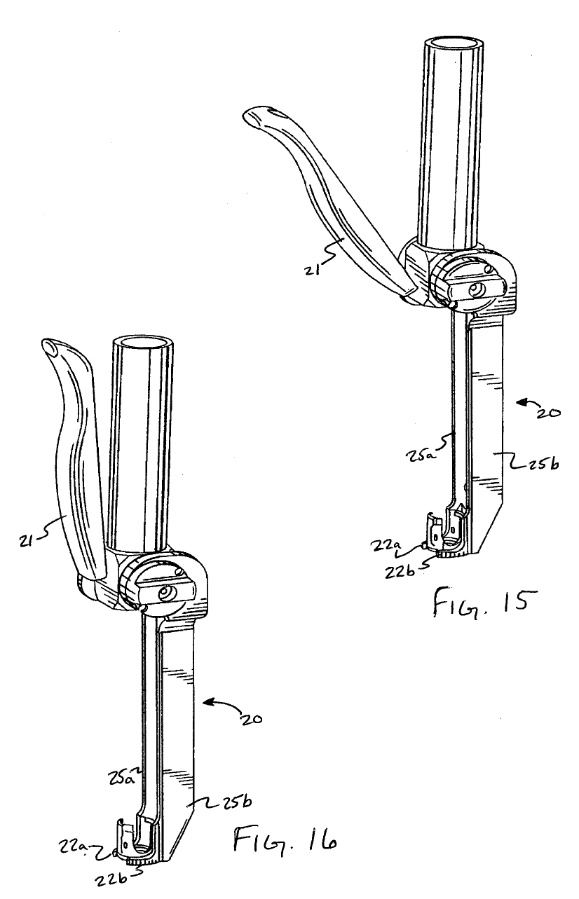

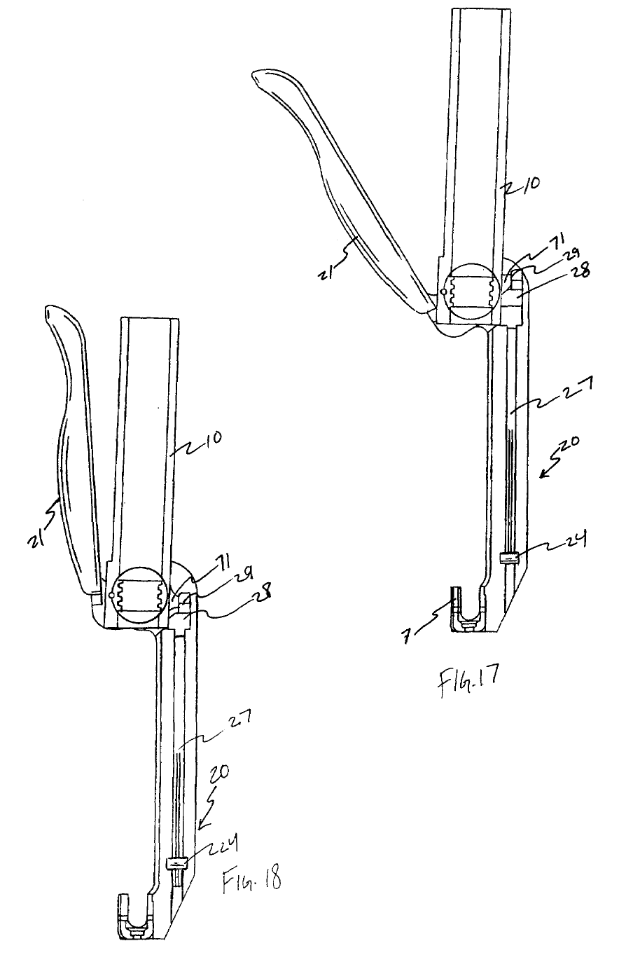

Clamping of the clamp device 20 to a coupling member having an anchor member mounted to the spine, such as the illustrated yoke 7, is accomplished by moving the clamping actuator lever 21 to clamp and unclamp the clamp device 20, as illustrated in FIGS. 15-20. When the actuator lever 21 is rotated upward, toward the ratchet handle 10, the flexible members 25a and 25b of the clamp device are drawn together by the movement of a plunger (27 in FIGS. 17-20) that is operated by the actuator lever 21. As the flexible members 25a and 25b are drawn together, a clamping force is provided sufficient to secure the yoke 7 between outwardly extending arms 22a and 22b connected to the flexible members.

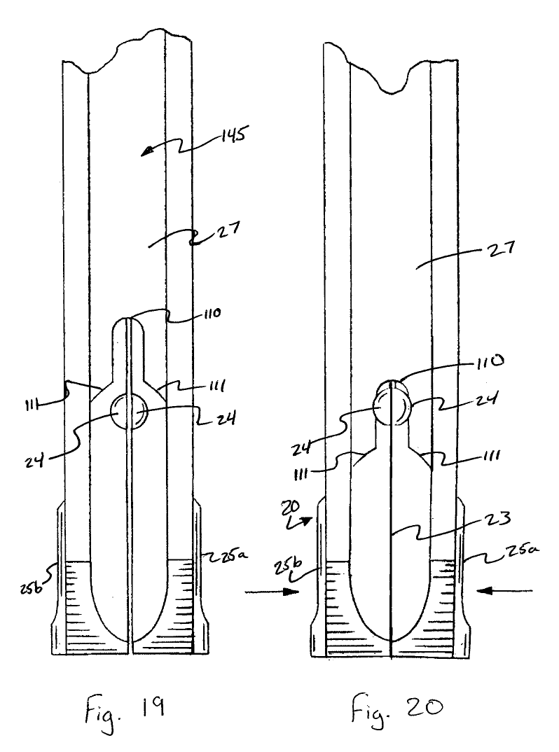

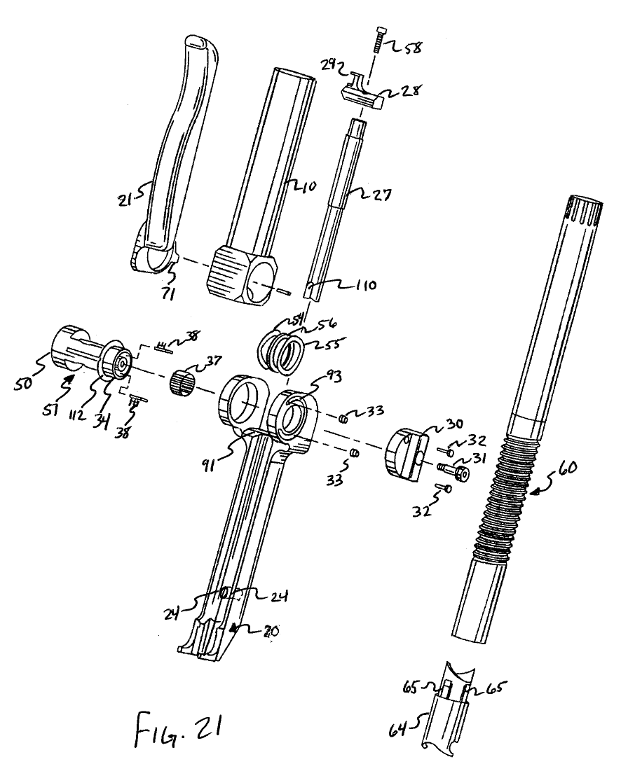

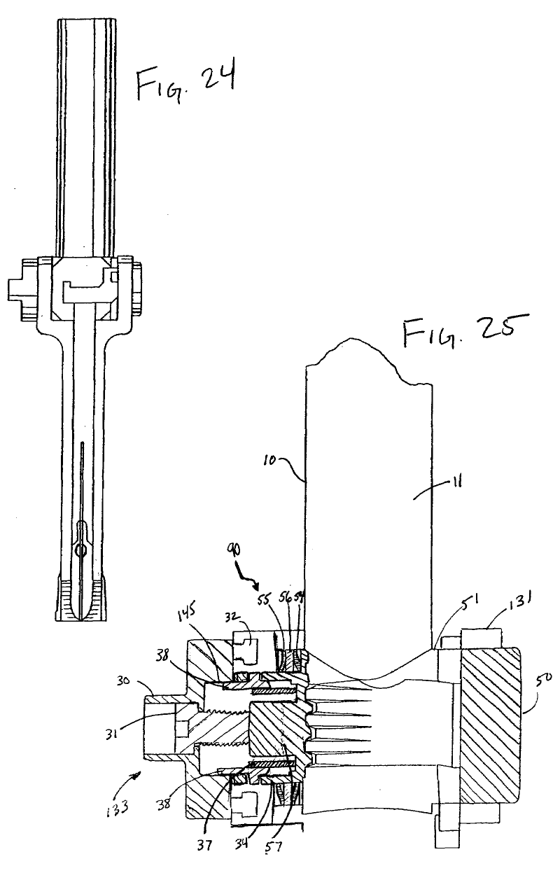

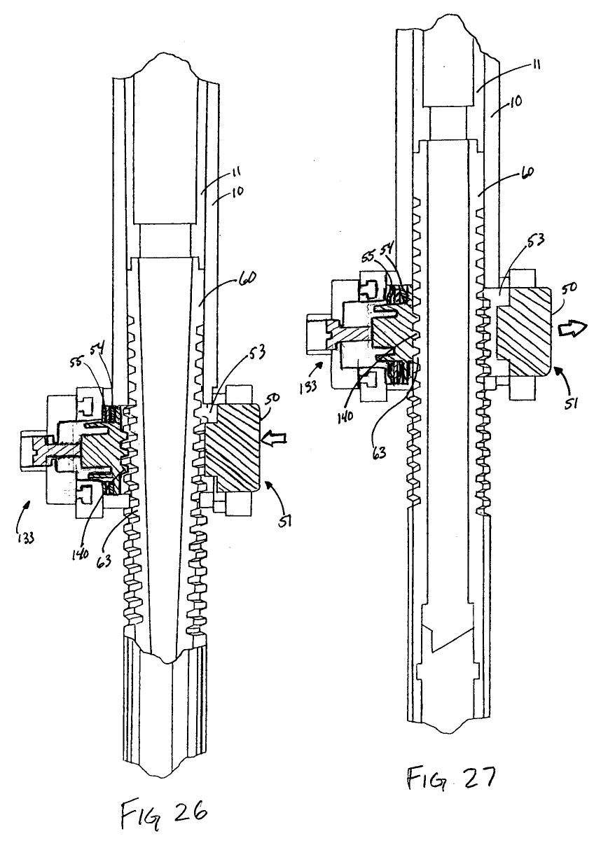

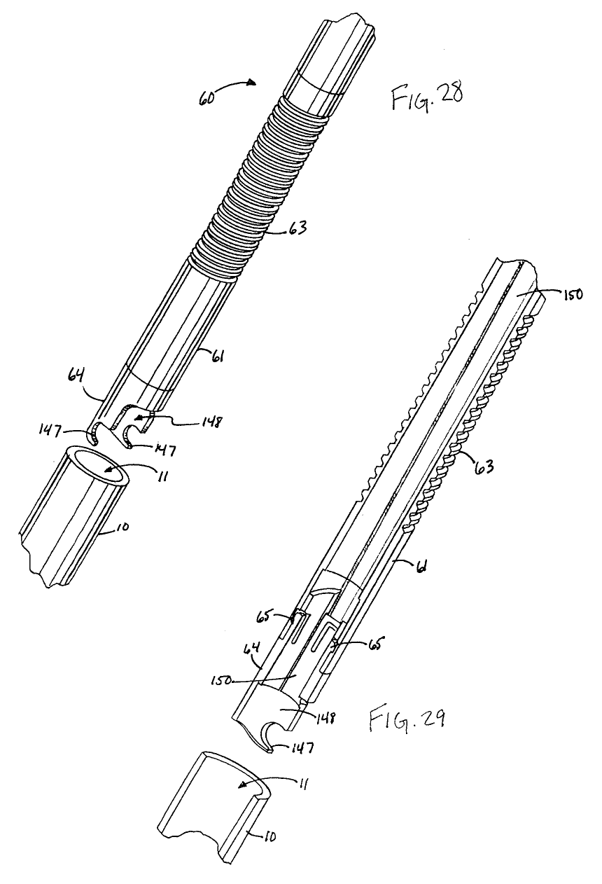

The side cross-sectional views in FIGS. 17 and 18, further illustrates the operation of the plunger 27 within the clamp device. As the clamp actuator lever 21 is moved toward the ratchet handle 10, a catch 71 extending radially from the axis of rotation of the lever 21 is moved down (clockwise in the view shown), engaging a catch 29 on a stop member 28, driving the stop member and attached plunger 27 downward through the hollow portion of the clamp device. Two laterally extending posts 24 traverse the hollow portion. As the plunger 27 moves axially through the hollow portion of the clamp device 20 and away from the central housing, a recess 110 at the bottom of the plunger engages both posts 24 and forces them together, drawing the flexible portions of the clamp device together and resulting in a clamping force sufficient to secure the yoke 7. Seen from a rear cross-section, as in FIGS. 19 and 20, as the plunger 27 is forced downward through an axial hollow portion 145 in the clamp device 20, inwardly-sloped surfaces 111 on the plunger 27 direct the two spaced-apart posts 24 traversing the hollow portion 145 toward a concave recess 110 in the plunger 27, as illustrated in FIGS. 19 and 20. The recess 110 is sized and configured to receive both posts 24 when spaced close together, but is not wide enough to receive the posts when spread apart in their normal spatial relationship. Therefore, when the plunger 27 is forced downward through the hollow clamp device, the plunger draws the posts 24 together, which in turn draws together the flexible legs 25a and 25b of the clamp, reducing or eliminating the width of the slit 23 formed between the flexible portions and resulting in a clamping action between the arms 22a and 22b. A full rear cross-section may be seen in FIG. 24.

Turning now to the details of the central operating assembly of the instrument 100 in FIG. 21, an exploded view of the main body and persuader arm of the instrument 100 display components of the clamp device 20, persuader arm 60, and central operating assembly 90 (including the ratchet assembly 133 and axial adjustment lock subassembly 59). As described previously, clamping actuator lever 21, ratchet handle 10, and clamp device 20, which is formed integrally with a central housing 91, are configured to be pivotally connected to each other. Assembly of the clamping actuator lever 21, ratchet handle 10, and clamp device 20 involves aligning a large aperture in each member along a common rotational axis and inserting a piston 51 through each of the aligned apertures to pivotally secure the members. The piston 51 also forms a portion of the central operating assembly, which actually comprises two separate subassemblies: the ratchet assembly 133 for fixing the angular position of the pivoting handle 10 and the axial adjustment lock subassembly 59 for engaging and disengaging the adjustable persuader arm 60, allowing the adjustable persuader arm 60 to slide into place within the hollow pivotable handle 10 and then become locked therein against sliding axial movement.

The ratchet assembly 133, which is described further with respect to FIGS. 22-23 comprises a ratchet dial 30 secured by a central bolt 31 and two smaller, radially spaced guide bolts 32, the ends of which are housed in retaining members 33 that are partially disposed in arcuate slots 93 of the housing 91. The central bolt 31 is partially threaded, so that the unthreaded portion nearest the head allows for smooth rotation of the ratchet dial 30 while the threaded portion of the central bolt 31 secures the ratchet dial to the assembly. The retaining members 33 help to hold the ratchet dial 30 to the housing 91 and limit rotation of the ratchet dial 30 to a predetermined angular value based on the size and configuration of the guide slots 92. The ratchet assembly 133 further comprises a cylindrical ratchet drum 34, inside of which is disposed a cylindrical radially expanding spring 37. In this case, the ratchet drum 34 is formed integrally with the piston 51. Camming ratchet teeth 38 are disposed radially outside of the radially expanding spring 37 and radially inside of the cylindrical ratchet drum 34, with the ends of the ratchet teeth extending radially outward through circumferentially opposed slots 39 in the ratchet drum 34.

The axial adjustment lock subassembly 59 comprises the piston 51 having a solid end forming an exterior depressible button 50 and also having an interior channel 53 situated transverse to the long axis of the piston. At least a portion of the interior of the transverse channel 53 is threaded. The axial adjustment lock subassembly 59 further comprises leaf springs 54 and 55 spaced by a washer 56 that serve to bias the piston 51 outward when it is not manually depressed by the surgeon.

With continued reference to FIG. 21, an actuating plunger 27 is provided that slides into the hollow portion of the clamp device 20 and translates linearly through the clamp in order to clamp and unclamp the flexible legs 25a and 25b of the clamp device 20, as described above. The actuating plunger 27 is moved linearly by movement of the clamping actuator lever 21, which contains a catch 71 that pushes against a corresponding catch 29 on a stop member 28 that is secured to the plunger 27 by a bolt 58. The plunger 27 has an arcuate recess 110 at its free end, the recess 110 being designed to engage and force together laterally extending posts 24 traversing the hollow portion of the clamp device 20, as demonstrated in FIGS. 19-20.

In one form, the persuader arm 60 is provided as a separate, elongate cylindrical member that may be disposed concentrically within the ratchet handle 10, which forms a guide portion or locking sleeve that allows the persuader arm 60 to slide axially and then be locked in place through use of the axial adjustment lock subassembly 59. The persuader arm distal engagement portion 64 contains a detent mechanism 65 for snap-locking to the persuader arm shaft 61 and being rotatable with respect thereto.

Further details of the central operating assembly 90, including the ratchet assembly 133 and axial adjustment lock subassembly 59, are shown in FIG. 22. Advantageously, the two subassemblies share certain components, which reduces the size and general profile of the instrument 100; facilitates assembly, disassembly, and cleaning; and economizes manufacture. As illustrated, the housing 91 for the assembly is formed integrally with the clamp device 20, but it will be readily understood that the housing may be formed separate from the clamp device. The housing 91 is located along the central operating axis 104 and is bounded on axial ends by collars 130 and 131, each having an aperture. The ratchet collar 130 forms part of the ratchet assembly 133. The opposed open collar 131 is wide enough to receive components of the two subassemblies.

The ratchet assembly 133 comprises a cylindrical ratchet drum 34. In the illustrated embodiment, the ratchet drum 34 is formed integrally with the piston member 51. Located circumferentially and coaxially inside the ratchet drum 34 lies a ratchet mounting member 57, to which a ratchet dial 30 is mounted when assembled.

Two sets of ratchet teeth 38 are provided in the ratchet assembly 133. The teeth are inserted into circumferentially-spaced slots 39 in the ratchet drum from the interior of the drum. Base portions 38a of the ratchet teeth form tabs that retain the teeth 38 in the slots 39. The base portions also extend slightly beyond the axial edge of the ratchet drum when the teeth 38 are disposed in the slots 39.

A radially expanding spring 37 is inserted concentrically and coaxially within the ratchet drum 34 and ratchet teeth 38, so that the exterior of the spring 37 contacts the bases of the ratchet teeth 38, biasing the teeth radially outward through the slots 39 in the ratchet drum. The spring 37 has a first diameter when less than a predetermined force is applied to its exterior, and is configured to contract radially to a second diameter when a radial force equal to or greater than the predetermined force is applied. In a resting state, the teeth protrude fully out of the circumferentially-spaced slots 39. However, when the spring 37 is compressed radially, the ratchet teeth 38 are allowed to retract into the circumferentially-spaced slots 39.

The ratchet drum 34, having been assembled with the ratchet teeth 38 and the radially expanding spring 37 so that the teeth and spring are held concentrically and coaxially within the drum 34, is inserted axially through the central operating housing 91 toward the ratchet collar 130 formed on the housing. The ratchet dial 30 approaches the ratchet collar 130 from an opposite direction, and is secured to the ratchet mounting member 57 using a mounting bolt 31. The mounting bolt 31 is threaded at one end so that it may be fixed within a threaded opening in the mounting member 57, but is unthreaded along a length of its shaft near the head in order to allow smooth rotation of the ratchet dial 30. The mounting bolt 31 extends through the aperture of the ratchet collar 130 in order to secure the ratchet dial 30 to the mounting member 37. The aperture in the ratchet collar 130 is of a sufficiently small diameter so that neither the piston 51 nor the ratchet dial 30 may pass through it, effectively confining the ratchet assembly 133 to the central housing 91.

When the ratchet assembly 133 is disposed in the central housing 91, the ratchet teeth 38 are aligned radially with housing teeth 92 located within the aperture of the ratchet collar 130. The ratchet teeth 38 may be extended and retracted radially to engage and disengage the housing teeth 92 by expansion and compression of the radially expanding spring 37 of the subassembly.

Two small guide bolts 32 are also disposed in the ratchet dial 30, radially spaced from the center of the dial and the mounting bolt 31 disposed therein. The guide bolts 32 pass through the ratchet dial 30 and attach to retaining members 33 that are configured to glide in arcuate slots 93 on the outward face of the housing collar 130. The paths of the arcuate slots 93 are dimensioned to provide limited rotation of the ratchet dial 30 from a discrete locked position to an unlocked position.

A recess 145 in the interior of the ratchet dial 30 is configured to radially retract and extend the ratchet teeth 38 when turned, as shown in FIG. 23. The perimeter of the recess 145 is non-circular when viewed along the rotation axis 104, so that the perimeter is narrower in a first radial direction than a second radial direction. In order to set the ratchet assembly 133 in an unlocked position, the ratchet dial 30 is rotated so that the narrow diameter of the recess is aligned with the tabs on the ratchet teeth 38a, applying a radial inward force that pushes the ratchet teeth 38 inward and compresses the radial spring 37. When the ratchet dial 30 is rotated so that a wide diameter of the camming recess 145 is aligned with the tabs 38a on the ratchet teeth 38 (the orientation shown in FIG. 23), the radial spring 37 is allowed to expand, pushing ratchet teeth 38 radially outward so that the tips of the teeth extend through openings in the ratchet drum 34 and engage complementary teeth on the ratchet collar 130 of the housing.

In discussing the ratchet assembly 133, reference to “clockwise” and “counterclockwise” will be made relative to the perspective shown in FIG. 23, which shows the side of the instrument 100 containing the ratchet dial 30. The ratchet assembly 133 operates by advancing ratchet teeth 38 from the assembly into engagement with complementary teeth 92 on the housing. When ratchet teeth 38 and housing teeth 92 are disengaged, free rotation of the ratchet drum 34 is permitted within the housing. However, the ratchet teeth 38 and housing teeth 92 are configured to permit rotation in only one rotational direction upon engagement, providing a ratchet lock between the housing and ratchet assembly 133. This ratchet lock is accomplished by providing one-way sloped tips on the ratchet teeth 38 that are complementary to one-way sloped tips on the housing teeth 92, so that the sloped tips slide past each other when the ratchet handle 10 is rotated counterclockwise with respect to the housing. However, the sloped tips of the complementary teeth resist rotation in the opposite direction, since parallel surfaces on the two sets of teeth abut upon attempted rotation in that direction.

The axial adjustment lock subassembly 59 will now be described with reference to FIG. 22. The axial adjustment lock subassembly 59 comprises leaf springs 54 and 55 separated by a washer 56, and a piston 51. The springs 54 and 55 shift the piston member along the operating axis 104 in order to lock the position of the extendable persuader arm 60 into place in the transverse channel 53 of the piston. The piston 51 is disposed within the central housing 91 of the instrument 100 along the operating axis 104. The base 120 of the pivotable ratchet handle 10 is also received in the housing 91, with the piston 51 being disposed through the transverse passage 115 through the handle base 120. Therefore, securing the piston 51 in the housing 91 simultaneously pivotably secures the handle 10 to the housing. The clamping actuator lever 21 is similarly held in the housing by the piston 51.

The open collar 131 opposite the ratchet collar 130 contains an aperture having a diameter large enough to slidably receive the piston 51. The central portion of the housing 91 is open from the top and side to receive the base 120 of the pivotable handle 10. As described above, the handle member 10 comprises a sleeve or axial guide. The base 120 of the handle member contains an open transverse channel 115 transverse to the cylindrical axis of the handle. The transverse channel 115 is aligned along the operating axis 104 within the central housing 91 during assembly, and slidably receives the piston member 51.

When the piston member 51 is received in the transverse channel 115 of the ratchet handle 10, the open channel 53 through the piston is aligned with the axial channel though the ratchet handle 10 so that a persuader arm may pass through both the handle and piston. When assembled, the persuader arm runs through the axial channel forming the hollow portion of the ratchet handle 10, through the opening 53 in the piston member, and exits an axial opening 116 at the bottom of the handle base 120. In order to align the opening 53 in the piston with the axial channel and axial opening 116 of the ratchet handle 10, an alignment recess 113 is formed along the exterior surface of the piston 51, with a corresponding alignment recess 117 on the interior of the transverse channel 115 of the handle member. When alignment recesses 113 and 117 face each other, the transverse passage 53 in the piston is aligned with the axial channel 11 of the ratchet handle 10. An alignment pin 114 is disposed between alignment recesses 113 and 117 in order to prevent rotation of the piston member 51 relative to the opening 116 in the handle base 120. The alignment pin 114 should be configured to prevent rotational movement, but to allow axial movement of the piston within the transverse channel 115 of the handle base 120. Alternatively, the piston may be rotationally fixed within the transverse channel of the handle portion by a ridge or groove formed on the surface of the piston that is complementary to a ridge or groove on the interior of the transverse channel of the handle, eliminating the need for the alignment pin 114.

The transverse throughbore 53 in the piston 51 is wider than the axial opening 116 of the handle 10 so that axial movement of the piston is permitted even when the persuader arm is disposed through the throughbore 53 of the piston and the axial handle base opening 116. One side of the transverse throughbore 53 through the piston contains threads configured to engage a threaded portion of the persuader arm. The piston member 51 is biased outward by leaf springs 54 and 55 so that the threads on the interior of the throughbore 53 in the piston 51 engage the persuader arm in a resting position. The leaf springs are separated by a washer 55 against which each leaf spring may compress and expand. The surgeon operating the instrument 100 may press in the end of the piston member 51, which forms a button slightly protruding from the housing 91, providing an axial force that pushes the piston member against leaf springs 54 and 55, causing the piston member 51 to shift toward the ratchet collar 130 and disengage the extendable persuader arm.

Referring now to FIG. 25, a cross-sectional view of the central operating assembly 90 is shown. The ratchet dial 30 is shown fixed to the mounting member 57 radially inward from a ratchet drum 34, with the ratchet drum 34 and dial 30 disposed on opposite sides of the ratchet collar 130 of the housing 91. The ratchet drum 34 is connected to the cylindrical piston 51, which is larger in diameter than the drum 34, forming a drum collar 112 on one side of the ratchet drum. Since the dial 30 and drum collar 112 are too large in diameter to pass through the housing collar 130, the ratchet assembly 133 is held in place within the housing along the rotational axis 104. Engagement and disengagement of the ratchet teeth 38 and housing teeth 92 is controlled by interaction between the cylindrical, radially expanding spring 37 and the recessed surface 145 on the interior face of the ratchet dial 30. The radially expanding spring 37 is disposed concentrically and coaxially within the ratchet drum 34 and biases the ratchet teeth 38 radially outward through openings in the ratchet drum 34. Tabs 38a extending axially from the ratchet teeth 38 contact the recessed interior surface 145 of the ratchet dial.

Turning now to the axial adjustment lock subassembly 59, shown in cross-section in FIG. 25, the piston 51 is aligned so that its transverse channel 53 is aligned with the ratchet handle throughbore 11. Leaf springs 54 and 55 bias the piston 51 axially outward through the open collar 131. The transverse piston throughbore 53 contains threads on one side of its interior.

The persuader arm 60 is inserted into the ratchet handle 10 and engaged and disengaged by the axial adjustment lock subassembly 59 to permit or restrict axial movement. The axial adjustment lock subassembly 59 is disengaged, as shown in FIG. 26, by manually depressing the external button 50 formed by the piston, shifting the entire piston 51 axially and compressing the leaf springs 54 and 55. When shifted, the persuader arm 60 may be slidably disposed in the axial channel 11 of the handle 10 and freely extended and retracted axially to a length required to engage a spinal rod, since the threads 140 in the transverse opening 53 of the piston are withdrawn from the path of the persuader arm, and do not engage a complementary threaded portion 63 located on the exterior of the persuader arm.

As shown in FIG. 27, when the external button 50 is no longer manually depressed, the leaf springs 54 and 55 located at the axially opposite end of the piston 51 shift the entire piston back outward, causing threads 140 on the interior of the transverse opening 53 through the piston 51 to engage the threaded portion 63 of the persuader arm 60 passing through the transverse opening 53, forming a drive mechanism. When the threads 140 on the interior surface of the piston throughbore engage the threads 63 on the persuader arm 60, the persuader arm 60 is prevented from sliding axially through the axial guide channel 11 of the ratchet handle 10. In this locked position, the persuader arm 60 may be adjusted axially only by rotating it with respect to the interior threads 140 of the piston 51. The persuader arm may therefore be incrementally retracted, pulling a spinal rod toward the instrument 100 against torsional forces from spinal deformities and the like, without worry that the persuader arm 60 will suddenly be extended. When the persuader arm 60 has been rotated to retract the arm a desired amount with respect to the ratchet handle 10, the surgeon may leave the rod in place without worry that the persuader arm will be pulled out of the axial handle channel 11.

By engaging and disengaging the axial adjustment lock subassembly 59, the persuader arm may be easily slidably extended to the length required to engage the laterally-displaced spinal rod, but also has the strength and ratcheting ability to incrementally draw the spinal rod linearly toward the instrument 100 in order to de-rotate the spine.

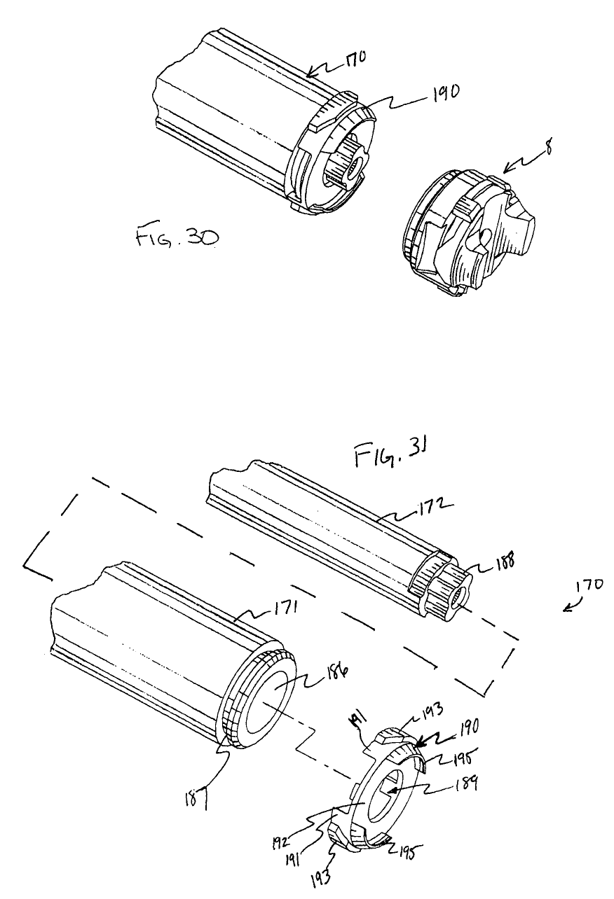

Turning now to details of the persuader arm engagement portion 64, as shown in FIGS. 28-29, axially spaced hook members 147 are configured to engage and capture the spinal rod. A hollow portion 148 is of sufficient size to receive the yoke into which the spinal rod will be loaded. The hooks 147 are spaced so that they will engage the spinal rod at axially spaced locations. The distance between the hooks 147 is greater than the diameter of the yoke, so that the hooks will not interfere with the loading of the rod into the yoke.

The persuader arm engagement portion 64 snaps into the shaft portion 61 and is held in place by detents 65 disposed in corresponding recesses on the interior surface of the persuader arm shaft portion 61, as shown in cross-section by FIG. 29. The engagement portion 64 is rotatable with respect to the rest of the persuader arm 60 so that the engagement portion 64 may maintain engagement with the rod as the persuader arm shaft 61 is rotated to produce linear travel, as described above.

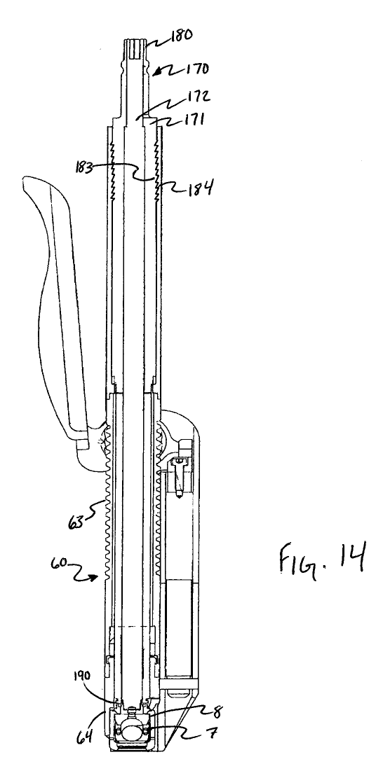

Turning now to details of the drive device 170, the inner shaft member 172 is disposed concentrically within the outer shaft member 171, which in turn may be inserted concentrically within the persuader arm 60, as shown in FIGS. 12-14. Radially outward extending bosses or flanges 193 on the bearing member 190 are sized to be received within facing axial slots 150 extending the length of the persuader arm passage. The flanges 193 permit the bearing member 190 to slide within the passage, but substantially hinder the rotation of the bearing member 190 as the outer shaft member 171 is rotated for linear travel through the interior of the persuader arm 60. Once the locking cap 8 has been at least partially inserted into the yoke, the flanges are free of the axial slots 150 so that the inner shaft member 172 is now able to rotate the locking cap relative to the yoke for further locking.

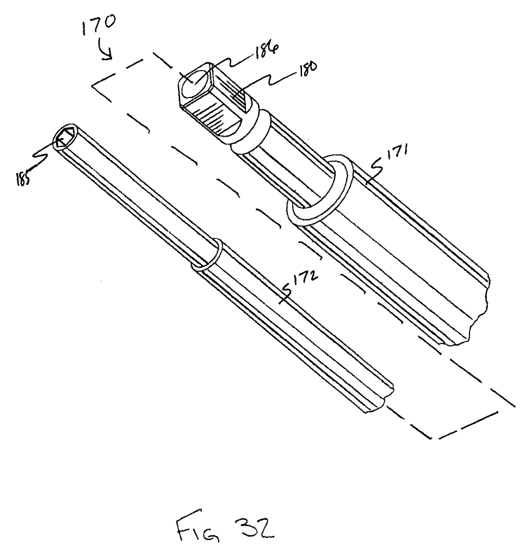

An exploded view of the drive device 170 is shown in FIG. 31. Extending through the outer shaft member 171, there extends an inner shaft member 172 having a distal tip 188 with a lobed profile arranged and configured to mate with a lobed recess on the top surface of a locking cap. Assembling the drive assembly 170 involves axially inserting the inner shaft member 172 into the passage 186 of the hollow outer shaft member 171, and then securing the bearing member 190 to the distal end of the outer shaft member 171 such that the tip 188 of the inner shaft member 172 may protrude through an aperture 189 in the bearing member 190 to engage the locking cap.

The bearing member 190 preferably comprises a disk-shaped base member 192 with a pair of facing, arcuate upstanding side walls 191 that extend axially upward from an outer edge of the base member 192. The base member 192 has a central opening 189 sized to permit the inner shaft member, and in particular the profiled tip 188, to extend therethrough. The bearing member 190 has an outer diameter permitting receipt within and passage through the persuader arm 60.

The bearing member 190 is preferably snap-fit onto the distal end of the outer shaft member 171. In one form, the outer shaft member 171 defines an annular ring 187 at its distal end that projects radially outward, and the bearing member 190 includes radial side walls 191 configured to snap onto the lip 187, such as by having an inwardly facing undercut groove complementary to the lip 187 on the outer shaft member 187. In order to receive the snap ring 187, the bearing member side walls 191 preferably resiliently flex outwardly and then snap back into their original configuration after the lip 187 has been received within the side walls 191.

The bearing member 190 is configured to hold the locking cap 8 in an interference or friction fit. In one form, the bearing member 190 preferably includes arcuate, lock fingers 195 that extend axially downward from opposite sides of the base member 192. Preferably, the lock fingers 195 are inclined or tapered radially inward towards the central opening 189 in order to receive and releasably secure the cap. In this manner, the drive device 170, and in particular the bearing member 190, is configured to retain the locking cap 8 thereon while being inserted into the passage through the persuader arm for insertion into the yoke member 7, but the drive device 170 may be easily removed from the cap assembly 8 after it has been locked into the yoke 7 by simply rotating the outer shaft member 171 in a reverse direction to retract the entire drive device 170. The press or friction fit of the cap assembly 8 to the bearing member 190 will be separated upon the reverse translation of the drive device 170 within the interior of the persuader arm. To this end, the bearing member 190 should be more tightly bound to the outer shaft member 171 than to the locking cap 8.

In order to advance and rotate the locking cap 8, the proximal end of both of outer shaft member 171 and inner shaft member 172 are configured to engage a rotating instrument 100, such as a wrench, for rotating its respective shaft. In this manner, when the inner shaft member 172 is inserted within the outer shaft member 171 to form the drive device 170, engaging and rotating a first portion of the drive device will linearly advance the locking cap secured to the distal end of the drive device, and rotation of a second portion of the drive device will cause rotation of the locking cap. FIG. 32 shows an exploded view of the proximal end of the drive device 170. The proximal end of the inner shaft member 172 preferably defines a profiled end 185, such as the hexagonal recess illustrated, that permits receipt of a rotating instrument 100 thereon. Likewise, the proximal end of the outer shaft member 171 preferably defines an exterior profiled end 180, such as the generally square head portion illustrated, that permits receipt of a different rotating instrument 100 thereon. In the embodiment shown in FIG. 32, the inner shaft member 172 is assembled into the passage 186 of the outer shaft member 171 so that the profiled end 185 of the inner shaft member is nested concentrically within the profiled head 180 of the drive device. In this manner, a first tool may be used to rotate the profiled head 180 of the drive device in order to drive a locking cap axially into a yoke to achieve one or more locked positions, and a second tool may be then used to engage and rotate the hexagonal recess 185 located within the profiled head 180 in order to affect rotation of the locking cap to a final locked position.

While there have been illustrated and described particular embodiments of the present invention, it will be appreciated that numerous changes and modifications will occur to those skilled in the art, and it is intended in the appended claims to cover all those changes and modifications which fall within the true spirit and scope of the present invention.

What is claimed is:

1. A surgical instrument for shifting a spinal rod toward a coupling member secured to a vertebra, the surgical instrument comprising:
a clamp device for clamping onto the coupling member;
a persuader arm having an engagement portion for capturing the rod and being pivotal relative to the clamp device for shifting the spinal rod toward the clamp device to position the rod above the coupling member; and
a guide portion pivotal relative to the clamp device and operable to guide the persuader arm for capturing of the rod by the engagement portion of the persuader arm.

2. The surgical instrument of claim 1, wherein the clamp device and guide portion have a ratchet mechanism therebetween configured to restrict pivoting of the persuader arm away from the clamp device and permit ratcheted pivoting of the persuader arm toward the clamp device.

3. The surgical instrument of claim 1, wherein the guide portion comprises a sleeve member through which the persuader arm is linearly shifted for positioning the engagement portion to capture the rod.

4. The surgical instrument of claim 3, wherein the persuader arm has exterior threads and wherein the persuader arm and the guide portion have a drive mechanism therebetween to selectively engage the threads of the persuader arm so that the persuader arm may be controllably advanced and retracted toward and away when the drive mechanism is in an engaged position and freely shifted through the guide portion when the drive mechanism is in a disengaged position.

5. The surgical instrument of claim 1, wherein the engagement portion of the persuader arm comprises a hook at a distal end thereof.

6. The surgical instrument of claim 1, wherein the distal end of the persuader arm has an axially extending opening sized to permit at least a portion of the coupling member to fit therein.

7. The surgical instrument of claim 1, wherein the instrument further comprises an axial throughbore extending through the persuader arm, and an elongate locking cap drive device sized to fit through the throughbore to drive a locking cap into engagement with the coupling member.

8. The surgical instrument of claim 7, wherein the cap drive device has outer and inner shaft members, with the inner shaft member disposed within and coaxial with the outer shaft member, the inner and outer shaft members being configured to allow for independent rotation therebetween.

9. The surgical instrument of claim 8, wherein the persuader arm and outer shaft member have a drive mechanism therebetween, so that the outer shaft member is shifted linearly relative to the persuader arm upon rotation of the outer shaft.

10. The surgical instrument of claim 8, wherein the inner shaft member has a drive end portion configured to engage a drive recess in the locking cap so that rotation of the inner shaft member causes rotation of the locking cap.

11. A surgical instrument for maneuvering a spinal rod toward a coupling member secured to a vertebra, the surgical instrument comprising:
a clamp device for clamping the coupling member;
a pivotal guide portion;
a persuader arm having a distal end configured to engage a spinal rod, the persuader arm extending through the guide portion and axially moveable therethrough; and
a locking cap drive device for driving a locking cap toward the spinal rod while the persuader arm is engaged with the spinal rod.

12. The surgical instrument of claim 11, including a lock device for selectively engaging the persuader arm to prevent axial sliding of the persuader arm with respect to the guide device.

13. The surgical instrument of claim 11, wherein the persuader arm has a hook member at a distal end thereof.

14. The surgical instrument of claim 11, wherein the distal end of the persuader arm has a recess or opening configured to receive the coupling member so that the distal end of the persuader arm may surround the coupling member while still engaged to the spinal rod.

15. The surgical instrument of claim 11, wherein the guide portion and persuader arm include a ratchet mechanism therebetween for controlled, incremental pivoting of the guide portion and persuader arm.

16. A method of shifting one or more vertebrae of a spine, the method comprising:
anchoring a coupling member to the vertebra;
securing a rod to the spine so that the rod is adjacent to the coupling member;
securing a clamp member to the coupling member;
pivoting an engagement portion of a persuader arm to capture the rod;
pivoting and axially shifting the persuader arm to maneuver the rod captured in the engagement end into the coupling member secured by the clamp member; and
driving a locking cap through an axial channel in the persuader arm in order to lock the rod to the coupling member.

17. The method of claim 16, wherein the locking cap is driven by rotating a threaded drive member in the axial channel of the persuader arm.

18. The method of claim 17, further comprising rotating the locking cap after it has been driven into the coupling member.

19. The method of claim 16, wherein pivoting of the persuader arm is accomplished with a rachet mechanism engaged so that pivoting of the engagement portion toward the coupling member is allowed but pivoting away from the coupling member is not allowed.

20. The method of claim 16, further comprising shifting the persuader arm axially without rotation to capture the rod.
